SEQUENCE LISTING (5 sequences)

SEQ ID NO: 1 - DNA, 4833 bp, Artificial Sequence
  pBB-CORA-200
gctcactcaaaggcggtaatctcgagtcccgtcaagtcagcgcgcaataaaaaagcccccggaaggtgatcttccgggggctttctcatgcgttccaagttatgtatggaccggccgacagatcgtcaagattactataagattccgcattgcggacgatttaggcgcactatcgagccatgctcccatttggcggagtttctttagatcccaaaaggcttgtcactacatcgccgatcagggttctcacttgtattaccagccgaaaaaataagctccaaaagcgtgactagatagcgctgtaaaccctaattttcctcggagaattggggtttttctttggttggtgatgatgatttttagagagatcgcctatgatcgagaccgttgaatttatggcagcaacatgaaacgggttcgggtcaagtacagttcaccagtcttttggctgggtcttggtttaaccggcattttgggcattggggcgatcgccaaccgcaatgctttacttcccttgtttccaggcagtggtcaggtggaatctccagcagcggtagattctgaagtgttagccctggtggatctcgccccggcagagcggcgcgatcgcctcgaagtgatcgccaacagcagcaacaatgacctcgaccgaaaccgcgcccgttacctgttggggatggactacttggttgcggaagatggggcagcggccctagccgccttcgagaacctagaacaggactatccggtgttaacgcctcatattttgattaaacggggccgcgcctacgaattggtcaataatcccgaacaggcccaggtgatttggtttgatgtggtgcaaaattatccagaggatgctgccgctgccgaagcgctgtttcgcctcagtgcctacgacccgaaatatgccgaccaggcgatcgccgaatatcccgcccatccgcgcacccaaagcttgatccaacaacgccttgcagaaagcccaactcaatcacaactcggcgttatgaatcttcggactcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagttatgagtcaggaaaaaaggcgacagagtaatctgtcgccttttttctttgcttgctttttgacacggtttagcagaaggacgtcgaacagtcatgaaattatggtggtgtcggatcagcgaacctcagttagcgtacattgtccaatctaactcaccggcgtgtcattgagggcttatttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagagaattacctaccggcctccagtactgtcatgtcgtcacggaacttccgattagaccctaaatttccagaattttgcctatttcgtctgattaataaggtaattgggaataggcagcctgggaaatagtcggcaactccgcatatttgccgaggatcgattgcaccattgcatagagacaagccactaaagtaccgagaaaggcaacatttgaaagggtttcaacgagaagactgccgcccccaaagccaaaaaagcgaattaaaatcccgaacaacaccagggcaatgtcaatcaaaatcgcctggagggtattgaagcgaatgaagtggctaattttgctgttgcgtaccaccgcagcatagagaatgaagaaaataatcagccctgcaaagggaaatgaataaaggcggattaggggttgtaaaggcacatagattaccgccaggatcggaaagcgcattaaaaacggcagaccgaagggcaaagcgtagacgaggggtaataggtaaggtaaggccccaaataaacgatctgtgggttcagtggtagtggacataaaactttttctgcaatggactagctcctattctacagagaagtcttgaagaagttctgtggcgatcgccagggccaaacaatgggaaaaattaaacgaaattcaggaattttgcggcgatttcccctgcatcctgacaaaaggcagtagaattgtaaacatttgttaatctctttttgaaactgaatgcaaagtacagtccgctccccagggagtcgcgagtctctacggcaagatctgcccttcaccctcaaggatgtgaaagcagccatcccagactactgttacagccgtagactacaacgggccacgcgtagtgagacatacgagttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatccggcaaaaaaacgggcaaggtgtcaccaccctgccctttttctttaaaaccgaaaagattacttcgcgttaaataagcacgtagtggcgcgcacttcagccaaggaagttgtcagtttcacctgttttacgtaaaaacccgcttcggcgggtttttacttttggggtgccacctgacgtctaagaaaccattattatcatgacattaacc

SEQ ID NO: 2 - DNA, 4531 bp, Artificial Sequence
  pBB-CORA-300
gctcactcaaaggcggtaatctcgagtcccgtcaagtcagcgcgcaataaaaaagcccccggaaggtgatcttccgggggctttctcatgcgttccaagttatgtatggaccggccgacagatcgtcaagattactataagattccgcattgcggacgatttaggcgcactatcgctctttttcctgggttgtggtgtcggcttactactgtcggtggtttgggtcaatgttgctcgccatagtcctccgctagaatcctccccagtcaaggtctcgcctcccttccaggtcgactagtcacaacaatttaaaaatcagaaaaattgtcccattgatcaacttacagggggccattgagcaaaatccggggtcaccatctagtccccaaaaagctggcgatcgccaaataatagtaaaacttatcattcaaatttaaaattacttagcagatccagggggacaactgcaaaattggtcggatttacatatagactttagcttatagatttcaagacataggcattcaaacctgcatagacaagagtctatacagagcgaagccaatggggttcattgcccctggaaagatcaagcaaactgccgaagattcagggccaagctttactaccccaatccccataaatttcaaccaaggagacaatttacattatggattttttgtccaatttcttgacggacttcgtgggacaattgcagtccccaaccctagcctttctgattggggggatggttattgccggcccaactcaatcacaactcggcgttatgaatcttcggactcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagttatgagtcaggaaaaaaggcgacagagtaatctgtcgccttttttctttgcttgctttttgacacggtttagcagaaggacgtcgaacagtcatgaaattatggtggtgtcggatcagcgaacctcagttagcgtacattgtccaatctaactcaccggcgtgtcattgagggcttatttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagagaattacctaccggcctccagtactgtcatgtcgtcacggaacttggcaatgcccactcctccacaggcggtgagggtaaagctcagcagtaaagttaccaggactacagcagtggatagggactttttcaacatgggagaaagggaagaactgggcaagaaggcaaaattacctttccttacccattaaacctccaatggttgaccagaaactagaggtagaatgattcccgccagaaaaagaagtttaacaatttgccatgggcactgttctgaagcggggaggaaggctatggcctcctcaaccaatttatttcctgcctaacccaggggacgttgttgaggctataagttgaggctataaatttaacttattaaaaggttcgacaaatttgagatagttttgtggcaaagatactgcttaggaaccaaatattgcataaacttagagatatagttttttctaaaaaaatagtcttatttctatctattgaatcggggcaatttaaactcagaatagattagttgttcccagctgaaaccatcgtgtgctttttccagaggcgtttttggcaatttttcctctggtaaatttcaccgactttggggcaatgctcataatcaccatagagtgaaatccatgaacaagtttgaatcaagacaatcggacagccgtagactacaacgggccacgcgtagtgagacatacgagttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatccggcaaaaaaacgggcaaggtgtcaccaccctgccctttttctttaaaaccgaaaagattacttcgcgttaaataagcacgtagtggcgcgcacttcagccaaggaagttgtcagtttcacctgttttacgtaaaaacccgcttcggcgggtttttacttttggggtgccacctgacgtctaagaaaccattattatcatgacattaacctactagtagcggccgctgcaggagtcactaagggttagttagttagccaattattgaagacgcttaacagcgtctttttttgtttctggtctccc

SEQ ID NO: 3 - DNA, 5326 bp, Artificial Sequence
  pBB-CORA-312
ctggaattcgcggccgcatctagagttgacaattaatcatccggctcgtataatgtgtggaattgtgagcggataacaatttcacacatactagagtagtggaggttactagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaactcggtaccaaattccagaaaagacacccgaaagggtgttttttcgttttggtcctactagtagcggccgctgcaggagtcactaagggttagttagttagccaattattgaagacgcttaacagcgtctttttttgtttctggtctcccgctcactcaaaggcggtaatctcgagtcccgtcaagtcagcgcgcaataaaaaagcccccggaaggtgatcttccgggggctttctcatgcgttccaagttatgtatggaccggccgacagatcgtcaagattactataagattccgcattgcggacgatttaggcgcactatcacccagatgggaaggtattttatgcttcctttgctgccgctgatgaccaagccacggatttaaccacggcgatcgccaatcccacggccatcgatttaattaacgccaggggatttacggcgggtagttccgtcaccgtatcgggttcctacagtcgggaagccttttttgatggatccatgggtttttatcgacttctggacgataacggtgcagtgctagatcccttaacaggtggtgtaatcaacccaggacaggtaggttatcaagaagcagctttggcagatagcaatcgtttgcaagccactggctccaccctaacggcagaagacctagaaaccagagcattttccttcaatattttgggtggcgagttgtatgcgccatttttaacggttaatgacagtctttccggtattaatcagacttattttgcctttgggtcggccaacccagatggcatcagccacagcacaaacttgggacccaacgtgattggttttgaagattttctcggcggaggcccaactcaatcacaactcggcgttatgaatcttcggactcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagttatgagtcaggaaaaaaggcgacagagtaatctgtcgccttttttctttgcttgctttttgacacggtttagcagaaggacgtcgaacagtcatgaaattatggtggtgtcggatcagcgaacctcagttagcgtacattgtccaatctaactcaccggcgtgtcattgagggcttatttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagagaattacctaccggcctccagtactgtcatgtcgtcacggaacttatgactattcaatacacccccctagccgatcgcctgttggcctacctcgccgccgatcgcctaaatctcagcgccaagagtagttccctcaacaccagtattctgctcagcagtgacctattcaatcaggaagggggaattgtaacagccaactatggctttgatggttatatgggaattcccggtatggatggcaccgatgcggaatcccaacagattgcctttgacaacaatgtggcctggaataacctgggggatttgtccaccaccacccaacgggcctacacttcggctattagcacagacacagtgcagagtgtttatggcgttaatctggaaaaaaacgataacattcccattgtttttgcgtggcccatttttcccaccacccttaatcccacagattttcaggtaatgcttaacacgggggaaattgtcaccccggtgatcgcctctttgattcccaacagtgaatacaacgaacggcaaacggtagtaattacagccgtagactacaacgggccacgcgtagtgagacatacgagttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatccggcaaaaaaacgggcaaggtgtcaccaccctgccctttttctttaaaaccgaaaagattacttcgcgttaaataagcacgtagtggcgcgcacttcagccaaggaagttgtcagtttcacctgttttacgtaaaaacccgcttcggcgggtttttacttttggggtgccacctgacgtctaagaaaccattattatcatgacattaaccttttgttatcaataaaaaaggccgcgatttgcggccttattgttcgtcttagttagttagcccttagtgactcgaattcgcggccgcttctagag

SEQ ID NO: 4 - DNA, 6082 bp, Artificial Sequence
  pBB-CORA-402
ctggaattcgcggccgcatctagagttgacaattaatcatccggctcgtataatgtgtggaattgtgagcggataacaatttcacacatactagagtagtggaggttactagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaactcggtaccaaattccagaaaagacacccgaaagggtgttttttcgttttggtcctactagtagcggccgctgcaggagtcactaagggttagttagttagccaattattgaagacgcttaacagcgtctttttttgtttctggtctcccgctcactcaaaggcggtaatctcgagtcccgtcaagtcagcgcgcaataaaaaagcccccggaaggtgatcttccgggggctttctcatgcgttccaagttatgtatggaccggccgacagatcgtcaagattactataagattccgcattgcggacgatttaggcgcactatcttccgcgatcgcccgtttacaagctgcctcagctggggcgatcacatcggcttgaagttgctgctgcacctgtttgatccgctcctgctgcacagggagttctgcttggctacgagcgacttcggtagcaatgtccgcttcagcttcggccaccaccgcttcgcgccgcgtcaacgcatcctgaatccggcgctcggcctcggcttgggcgatcgctacatcgcgatcgatccgacgcagggccgtgatcttgtcattttcggccgtttggatcgcagaggcagcctgggcatcggcttcagcaattcgggcatctcgctgcagatcagcccgctgcttgcgtccactagccgagagataaccgacctcatcggaaatgttctggacttgcagcgtatcgaggactagacccagctgctcaaggtcatcctccgcctcttccagcagacttttggcaaaggcaattttgtcctcgttgatctgctccggcgtgaggctggctaaaacaccacgcaagttgccttcgagggtctccttggcaatttgctcgatttccttacggtttttgccaagcagccgctcgatcgcgttgtggatggtcggttcttccccagcaatcttgatattggcaacgccttcaacagtcaggggaatgccgcccttggagaaggcattggaaacgcgcaactcaatgatcatgttggtcagatccatgcggagcgctttttccagcagaggtacccgcaggctgctgccgcccttgaccaagcgatagccaactcggcggccatcactactgcggcgactactgccagcaaagatcaaaatttcactgggttggcagatgtagtagagattgcgcaggactaagctgccagccccggcggcccaactcaatcacaactcggcgttatgaatcttcggactcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagttatgagtcaggaaaaaaggcgacagagtaatctgtcgccttttttctttgcttgctttttgacacggtttagcagaaggacgtcgaacagtcatgaaattatggtggtgtcggatcagcgaacctcagttagcgtacattgtccaatctaactcaccggcgtgtcattgagggcttatttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagagaattacctaccggcctccagtactgtcatgtcgtcacggaacttgatccggcagccggcggagcgctgctttcttggcaagcggtcgccagccccaacgccagggctgccagcccgaaacagcggggcaaggcagcttggaagggcgatcgcagcacgggcatggcaatgtctctctgaaggaatgcagaccttattcgtacagccagggttgaatcgtgggggtccaatcacttagctctgctgggctaaaccagagagcaatttcctgttgtgctgtttcgattgcatccgagccatggatgatgttgcggccaatattgacaccaaaatcaccacggatggtgcccggttctgccgtcagcggattggtagcgccgatcaacttgcgagcagccgccacaacgccttcgccttccaagacgatcgccacgatcggcccagaggtgatgaactcgacgaggccattgaagaaggggcgctcgcggtggacagcatagtgctgttcggccagctcgcgactgggcttcagctgctttaggcccaccagtttgaagcctttttgctcaaagcggccgatgatcgtaccgaccaaaccccgctgaacgccatcgggcttgatggcaataaatgtgcgttccacagacatctagatagtcctcaagacgaggcaagcattgagcttgccttcctatggttcgggatcactgggattcttgacaagcgatcgcggtcacatcgctatctcttaggacttcgcagcgggcgagtcggattgacccggtagggatttcgccagatcaatgcccgtggtttgtttcagcttctccagcaagctagcgatttgggtagcgctgccttccccttcgccaatcacagtgatcgactccacgtcgatatctggcacggtgcctgaaagcgtgacgagcagggactcgacagccgtagactacaacgggccacgcgtagtgagacatacgagttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatccggcaaaaaaacgggcaaggtgtcaccaccctgccctttttctttaaaaccgaaaagattacttcgcgttaaataagcacgtagtggcgcgcacttcagccaaggaagttgtcagtttcacctgttttacgtaaaaacccgcttcggcgggtttttacttttggggtgccacctgacgtctaagaaaccattattatcatgacattaaccttttgttatcaataaaaaaggccgcgatttgcggccttattgttcgtcttagttagttagcccttagtgactcgaattcgcggccgcttctagag

SEQ ID NO: 5 - DNA, 4757 bp, Artificial Sequence
  pBB-CORA-410
gctcactcaaaggcggtaatctcgagtcccgtcaagtcagcgcgcaataaaaaagcccccggaaggtgatcttccgggggctttctcatgcgttccaagttatgtatggaccggccgacagatcgtcaagattactataagattccgcattgcggacgatttaggcgcactatcggtaaccccagcgcggttgctaccaagtagtgacccgcttcgtgatgcaaaatccgctgacgatattcgggcgatcgctgctgaatgccatcgagcagtaacgtggcaccccgcccctgccaagtcaccgcatccagactgaacagcaccaagaggctaaaacccaatcccgccggtagcagcggagaactacccagcattggtcccaccaaagctaatgccgtcgtggtaaaaatcgcgatcgccgtcagactcaagcccagttcgctcatgcttcctcatctaggtcacagtcttcggcgatcgcatcgatctgatgctgcagcaagcgttttccataccggcgatcgcgccgtcgccctttcgctgccgtggcccgcttacgagctcgtttatcgaccacgatcgcatccaaatccgcgatcgcttcccagtccggcaattcagtctggggcgtccgtttcattaatcctgatcaggcacgaaattgctgtgcgtagtatcgcgcatagcggccagcctctgccaacagcgcatcgtgattgcctgcctcaacaatctggccgcgctccatcaccaagatgcggctggcattacgaaccgtagccagacggtgagcaatgataaagaccgtccgtccctgcatcacccgttctagggcctcttgcaccaaggtttcggactcggaatcaagcgccgaagtcgcctcatccagaattaaaatgcgtggatcgcccaactcaatcacaactcggcgttatgaatcttcggactcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagttatgagtcaggaaaaaaggcgacagagtaatctgtcgccttttttctttgcttgctttttgacacggtttagcagaaggacgtcgaacagtcatgaaattatggtggtgtcggatcagcgaacctcagttagcgtacattgtccaatctaactcaccggcgtgtcattgagggcttatttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagagaattacctaccggcctccagtactgtcatgtcgtcacggaacttcgtctgcaagaagccggtgcccatctcatttttgacgatatgcgactgctgcccagtctgctccaatcgtcgccaaaagataactccacagcattgcccaatccctaacccctgctcgcgccgcaactacacactaaaccgttcctgcgcgatcgctcttactgttgatggctcgtgcttaaaaacaatgcaaccctaaccgtttcagctggtgattttcggacgatttggcttacagggataactgagagtcaacagcctctgtccgtcattgcacacccatccatgcactggggacttgactcatgctgaatcacatttcccttgtccattgggcgagaggggaggggaatcttctggactcttcactaagcggcgatcgcaggttcttctacccaagcagtggcgatcgcttgattgcagtcttcaatgctggcctctgcagccatcgccgccaccaaagcatcgtaggcgggacgttgttgctccagtaaagtcttcgcccgtaacaatccccagcgactgcgtaaatccgcttcggcaggattgcgatcgagttgccgccacagttgtttccactgggcgcgatcgtcagctcccccttccacgttgccgtagaccagttgctctgccgctgcaccggccatcaacacctgacaccactgttccagcgatcgctgactgagttgcccctgtgcggcttcggcttctagcgcagctgcttggaactgcacacccccgcgaccaggttgtccttggcgcagcgcttcccacgctgagagggtgtagcccgtcacgacagccgtagactacaacgggccacgcgtagtgagacatacgagttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatccggcaaaaaaacgggcaaggtgtcaccaccctgccctttttctttaaaaccgaaaagattacttcgcgttaaataagcacgtagtggcgcgcacttcagccaaggaagttgtcagtttcacctgttttacgtaaaaacccgcttcggcgggtttttacttttggggtgccacctgacgtctaagaaaccattattatcatgacattaacc

(19) United States
(12) Patent Application Publication
(10) Pub. No.: US 2023/0183714 A1
(43) Pub. Date: Jun. 15, 2023

(54) METHODS FOR TRANSFORMING CYANOBACTERIA

(71) Applicant: BONDI BIO PTY LTD, Sydney (AU)

(72) Inventors: James BROWN, Sydney (AU); Dominik KOPP, Sydney (AU); Cameron O'BRIEN, Sydney (AU)

(21) Appl. No.: 17/915,871

(22) PCT Filed: Apr. 1, 2021
(86) PCT No.: PCT/AU2021/050300

§ 371 Date: Sep. 29, 2022

(30) Foreign Application Priority Data
Apr. 3, 2020 (AU) 2020901047

(51) Int. Cl. C12N 15/65; C12N 1/20; C12N 15/74

(52) U.S. Cl. CPC C12N 15/65; C12N 1/20; C12N 15/74; C12N 2800/101

(57) ABSTRACT

The technology relates to a method for producing a transformed and fully-segregated cyanobacteria, the method comprising incubating the cyanobacteria and a nucleic acid comprising a selectable marker under conditions suitable for transformation of the cyanobacteria with the nucleic acid; further incubating the cyanobacteria in growth media under conditions suitable for recovery of the cyanobacteria; and selecting the transformed and fully-segregated cyanobacteria using a selection agent.

### TECHNICAL FIELD

The technology relates to methods of transforming Cyanobacteria to produce transformed and fully-segregated cyanobacteria.

### CROSS REFERENCE TO RELATED APPLICATION

This application claims the benefit of Australian Provisional Application No. 2020901047 filed 3 Apr. 2020, the entire content of which is incorporated by reference herein.

### BACKGROUND

Cyanobacteria produce a large number of secondary metabolites and have the potential to be used in the production of pharmaceuticals, high value chemicals and as tools for bioremediation. The diverse array of biochemical pathways of Cyanobacteria are apparent in the more than 400 cyanobacterial genomes available in public databases (Alvarenga et al, Front. Microbiol., volume 8, 2017, page 809). Consequently, the potential to improve and/or modify metabolite production by employing genetically manipulated cyanobacteria is being explored.

Currently, three procedures are available to introduce heterologous nucleic acid into cyanobacteria: natural transformation, electroporation, and conjugation. The success of transformation using each method is relatively low and may be species dependent due to the different physical and biochemical barriers against foreign nucleic acid insertion across different species. Further, the transformation success may depend on the length, form and concentration of the nucleic acid used.

Integrative and replicative vectors can be used to transform cyanobacteria. Integrative plasmids incorporate heterologous nucleic acid into genomic DNA by homologous recombination. Alternatively, replicative plasmids allow the introduction of heterologous nucleic acid and are capable of self-replicating in the cell. Both types of plasmids have been developed for cyanobacteria.

The genetic manipulation of cyanobacterial strains is challenging due to the inefficiency and laborious nature of current transformation protocols which typically requires a period of 3-6 months in order to produce pure stains of genetically manipulated and fully segregated cyanobacteria.

Cyanobacteria have variable ploidy with some strains having 3-4 genome copies per cell and others having 218 genome copies in exponential phase and 58 genome copies in linear and in stationary growth phase. That is, the ploidy level is highly growth phase-regulated. Further, cell division and segregation are not temporally separated and so segregation occurs progressively following replication. A consequence of this is that when a cyanobacteria is transformed with a nucleic acid, the genome copy carrying the transformed nucleic acid often does not fully segregate into subsequent generations.

Accordingly, the development of new transformation protocols is desirable to facilitate the genetic engineering of cyanobacteria.

The present inventors have developed methods for the production of fully-segregated stains of genetically manipulated Cyanobacteria in as few as 7-9 days post transformation. The methods significantly reduce the resources and time required for production of genetically manipulated Cyanobacteria.

### SUMMARY

In a first aspect, there is provided a method for transforming a gram negative micro-organism, the method comprising;
a) incubating the micro-organism and a nucleic acid comprising a selectable marker under conditions suitable for transformation of the micro-organism with the nucleic acid;
b) further incubating the micro-organism in growth media under conditions suitable for recovery of the micro-organism; and
c) selecting the transformed micro-organism using a selection agent.

The gram negative micro-organism may be a cyanobacteria, for example a cyanobacteria of the genus Synechococcus or Synechocystis. The Synechococcus may be Synechococcus sp. PCC 7002, or Synechococcus elongatus PCC 7942. The Synechocystis may be Synechocystis sp. PCC 6803.

In one embodiment the transformed micro-organism is fully-segregated.

The cyanobacteria in step a) may be in exponential growth phase. In some embodiments of the step prior to step a), the cyanobacteria may have been cultured in light/dark cycles. The cyanobacteria used in step a) may be harvested at or near the end of a light cycle.

The conditions suitable for transformation may comprise incubating the cyanobacteria for a period of 1-10 hours under low light conditions, for example about 5 hours.

The conditions suitable for recovery may comprise adding growth media and incubating the cyanobacteria for about 1 to about 24 hours under low light conditions, for example about 4 to about 18 hours.

The selecting step may comprise adding a selection agent and incubating the cyanobacteria for about 12 to at least about 144 hours under low light conditions, for example about 48 hours to about 144 hours.

The incubation, further incubation or both may be performed in aqueous media.

In some embodiments a portion of the cyanobacteria in the selecting step may be applied to a solid or semi-solid media after the incubation period to obtain individual colonies.

In a second aspect there is provided a transformed cyanobacteria produced by the method of the first aspect.

In a third aspect there is provided a method for transforming a cyanobacteria, the method comprising;
a) incubating the cyanobacteria and a nucleic acid comprising a selectable marker for a period of 1-10 hours under low light conditions;
b) further incubating the cyanobacteria in growth media for about 1 to about 24 hours under low light conditions; and
c) selecting the transformed cyanobacteria using a selection agent, wherein the selecting comprises adding the selection agent and incubating the cyanobacteria for about 12 to at least about 144 hours under low light conditions.

In a fourth aspect there is provided a method for transforming a cyanobacteria, the method comprising;
a) incubating the cyanobacteria and a nucleic acid comprising a selectable marker for a period of about 5 hours under low light conditions;
b) further incubating the cyanobacteria in growth media for about 4 to about 18 hours under low light conditions; and
c) selecting the transformed cyanobacteria using a selection agent, wherein the selecting comprises adding the selection agent and incubating the cyanobacteria for about 48 to about 144 hours under low light conditions.

The cyanobacteria may be in an exponential growth phase.

In one embodiment the incubation, further incubation or both are performed in aqueous media.

Preferably the transformed cyanobacteria is fully segregated.

### Definitions

Throughout this specification, unless the context clearly requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term ‘consisting of’ means consisting only of.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms ‘a’ and ‘an’ are used to refer to one or more than one (i.e., at least one) of the grammatical object of the article. By way of example, reference to ‘an element’ means one element, or more than one element.

In the context of the present specification the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

In general the process of segregation refers to the process of chromosome segregation which is the process during which sister chromatids formed as a consequence of DNA replication, or homologous chromosomes present in an oligoploid or polyploid cyanobacteria, separate from each other and migrate to different parts of the cyanobacteria such that when the cell divides each daughter cell receives at least one copy of the sister chromatid or homologous chromosome. As used herein ‘segregation’ additionally refers to a process where a selection pressure is applied to a cyanobacteria transformed with a nucleic acid. The selection pressure creates a survivorship bias such that only cyanobacteria containing at least one copy of the nucleic acid survive.

‘Fully segregated’ is used herein in reference to a cyanobacteria transformed with a nucleic acid wherein the transformed nucleic acid is present in multiple generations of the cyanobacteria such that substantially every individual cyanobacteria in a culture comprises the transformed nucleic acid. A ‘fully segregated’ cyanobacteria is a cyanobacteria transformed with a nucleic acid targeted to a neutral site wherein the nucleic acid is present in the targeted neutral site in substantially every chromosome or plasmid within each individual cyanobacteria and no copies of the original, unmodified chromosome are present.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

In order that the present technology may be more clearly understood, preferred embodiments will be described with reference to the following drawings and examples.

### BRIEF DESCRIPTION OF THE FIGURES

FIG. 1 is a plasmid map of CORA-312.

FIG. 2 is a plasmid map of CORA-200.

FIG. 3 is a plasmid map of CORA-410.

FIG. 4 is a gel showing fully segregated transformants of CORA-312 and CORA-200.

FIG. 5 is a plasmid map of CORA-402.

FIG. 6 is a gel showing fully segregated transformants of CORA-402.

FIG. 7 is a plasmid map of CORA-300.

FIG. 8 is a gel showing fully segregated transformants of CORA-300.

### SEQUENCE LISTING

A listing of nucleotide sequences corresponding to the sequence identifiers referred to in the specification is provided. The nucleotide sequence of plasmid pBB-CORA-200 is set forth in SEQ ID NO: 1. The nucleotide sequence of plasmid pBB-CORA-300 is set forth in SEQ ID NO: 2. The nucleotide sequence of plasmid pBB-CORA-312 is set forth in SEQ ID NO: 3. The nucleotide sequence of plasmid pBB-CORA-402 is set forth in SEQ ID NO: 4. The nucleotide sequence of plasmid pBB-CORA-410 is set forth in SEQ ID NO: 5.

### DESCRIPTION OF EMBODIMENTS

### Methods

There is provided a method for transforming and obtaining fully-segregated clones of transformed cyanobacteria. The methods comprise the steps of providing cells at a particular phase of growth, contacting the cells with a nucleic acid, incubating the cells with the nucleic acid for a period, allowing the cells to recover with additional growth media for a period before adding a selection pressure to select fully-segregated clones of transformed cyanobacteria.

### Cell Growth and Preparation

Actively growing cyanobacteria are used in the transformation methods. The cyanobacteria may be in early, mid or late exponential phase. This can be determined using an OD measurement, for example at 750 nm. Cyanobacteria from a culture with an OD of 0.1 to 3.0 can be used. For example suitable ODs are 0.1, 0.2, 0.4, 0.6, 0.8, 1.0, 1.2, 1.4, 1.6, 1.8, 2.0, 2.2, 2.4, 2.6, 2.8, or 3.0.

Cyanobacteria cultured under any growth conditions known in the art can be used. In some embodiments the cyanobacteria are grown under low-light conditions, constant light or using periods of light and dark, for example light and dark periods that mimic a normal day/night cycle.

In embodiments where a light/dark cycle is used to prepare the cyanobacteria for transformation, the cyanobacteria may be harvested at any point in the light/dark cycle. However, it is known that in some (but not necessarily all) strains pilus biogenesis occurs daily in the morning, but natural competence is at its peak with the onset of darkness, that is natural cyanobacterial competence is conditional and tied to the cells’ circadian rhythm. Accordingly, in some embodiments the cells are harvested at or near the transition from light to dark, or near the end of the light cycle.

The cyanobacteria cultured for transformation may be cultured in low-light conditions (i.e. less than 100 µmol photons · m⁻² · s⁻¹), for example 50 µmol photons · m⁻² · s⁻¹, normal light conditions (from 100 - 750 µmol photons · m⁻² · s⁻¹), for example 100-150 µmol photons · m-2 · S⁻¹ or light saturated conditions (greater than 750 µmol photons · m⁻² · s⁻¹). In embodiments where light/dark cycles are used the level of light in each light cycle may be independently selected from low-light, normal light or light saturated.

Broad spectrum light is typically used however it is envisaged that light comprising various wavelengths and irradiance levels may also be used, that is the total amount of light energy available at the wavelengths (or a range of wavelengths) can be adjusted to optimise or modulate cell growth and/or cell function.

In some embodiments the cells are harvested by centrifugation. Alternatively, the cells may be harvested by filtration, sedimentation or any other methods known in the art. Although not essential, the harvested cells are typically washed with a solution that is free of growth media, such as 10 mM NaCl.

In one embodiment the harvested cells are resuspended in fresh growth media to a concentration of approximately 10⁹ cells per mL. The concentration of resuspended cells may be from 10⁵ cells per mL to at least 10¹² cells per mL.

Aliquots of the resuspended cells are dispensed to suitable containers (such as PCR tubes or wells of a multi-well plate) for transformation. The present inventors have demonstrated that 20 µL aliquots provide cells although it is envisaged that any aliquot volume may be used, for example 10 µL.

In some embodiments the cells are grown without controlling CO₂ levels. In other embodiments the cells are cultured in an atmosphere comprising about 0.05% to about 10% CO₂, for example the CO₂ level is about 0.05%, 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% or about 10%.

### Contacting Cells with Nucleic Acid

The cyanobacteria are then contacted with the nucleic acid to be transformed. For example, by either adding the nucleic acid to the dispensed cells or the nucleic acid may already be present in the containers when the aliquots are dispensed. In some embodiments 100 ng of nucleic acid is used. In other embodiments 1 to at least 500 ng nucleic acid may be used per 20 µL of aliquot. For example, about 1 ng, 5 ng, 10 ng, 50 ng, 100 ng, 150 ng, 200 ng, 250 ng, 300 ng, 350 ng, 400 ng, 450 ng, or at least 500 ng or nucleic acid may be used per 20 µL of aliquot.

### Nucleic Acid

Any type of nucleic acid may be used, for example linear or circular DNA.

The nucleic acid can be used in the methods disclosed herein to make genetic modifications to a cyanobacteria. Such modifications can be made either in cis (e.g. by chromosome modification) or in trans (e.g. by the addition of a plasmid, for example a plasmid that is used to modify a plasmid naturally found within a cyanobacteria).

Cis genetic modification is typically used to modify a cyanobacterial chromosome as it takes advantage of the capability of many cyanobacterial strains for natural transformation and homologous recombination in order to create an insertion, deletion, or replacement mutations in the cyanobacterial chromosome. In some embodiments strains are transformed with selectable markers (such as an antibiotic resistance gene) and a sequence of interest, wherein the selectable marker and sequence of interest are flanked by sequences homologous to any non-essential sequence on the chromosome. This can take the form of a suicide vector designed to integrate into the genome at a non-essential site due to the flanking regions in the vector. The vector will also contain an insert comprising a sequence of interest and optionally a selectable marker.

Suitable antibiotic resistance genes confer resistance to chloramphenicol, erythromycin, kanamycin, spectinomycin, neomycin, streptomycin, zeocin or gentamicin

The sequence of interest may be for example a modified version of one or more cyanobacterial genes or may be one or more heterologous genes to be expressed in the cyanobacteria.

Alternatively there may be no sequence of interest and the selectable marker with flanking sequences either side may be used to delete a portion of the cyanobacterial genome, for example to knock out a gene or portion thereof.

In some embodiments the sequence of interest may not contain a gene or genes to be expressed but rather may merely comprise a selectable marker. In these embodiments the flanking regions are designed to be homologous to a portions of the cyanobacterial genome either side of a region that is to be deleted by the suicide vector and replaced by the selectable marker.

Alternatively, markerless mutants can be made either by selection-counter selection or by using a recombinase system such as FLP/FRT.

The counter-selection method begins with using a suicide vector as set out above but the insert also contains a counter-selectable marker such as sacB. In these methods the counter-selectable marker such as sacB (a conditionally toxic gene) is linked to a selectable marker such as antibiotic resistance cassette and then this plasmid is transformed into the cyanobacteria using the methods described herein, with selection for antibiotic-resistant mutants. A second transformation is carried out in which the resistance cassette and toxin gene are deleted, and markerless mutants are selected which have lost the toxic gene.

A suitable counter selectable marker is the B. subtilis levansucrase synthase gene sacB, which confers sucrose sensitivity. Alternatively the E. coli mazF, protein synthesis inhibitor expressed under a nickel-inducible promoter can also be used. This allows the reuse of a single selectable marker for making multiple successive changes to the chromosome. Other suitable counter selectable markers include rpsL, tetAR (confers sensitivity to fusaric and quinalic acids), pheS (confers sensitivity to p-chlorophenylalanine), thyA (confers sensitivity to trimethoprim and related compounds), lacY (confers sensitivity to sensitive to t-o-nitrophenyl-β-d-galactopyranoside), gata-1 (inhibits the nucleic acid replication), and ccdB (a toxic protein).

The flanking regions can be designed to be homologous to any region of the cyanobacterial genome and a skilled person can design the flanking regions using methods known in the art.

In some embodiments the length of the flanking regions are at least 500 bp. For example, the length of each flanking region may be independently selected from about 500 bp, 550 bp, 600 bp, 650 bp, 700 bp, 750 bp, 800 bp, 850 bp, 900 bp, 950 bp or at least about 1000 bp.

The flanking regions may be homologous to any region of the cyanobacterial genome. In some embodiments the flanking regions are homologous to non-essential regions. Non-essential regions are known in the art.

For example suitable non-essential regions for PCC 6803 are described as the NSC1 site by Ng, A.H., Berla, B.M. and Pakrasi, H.B., 2015. Fine-tuning of photoautotrophic protein production by combining promoters and neutral sites in the cyanobacterium Synechocystis sp. strain PCC 6803. Appl. Environ. Microbiol., 81(19), pp.6857-6863.

In another embodiment the non-essential site may for PCC 6803 may be slr0168 as described by the Xiao, Y., Wang, S., Rommelfanger, S., Balassy, A., Barba-Ostria, C., Gu, P., Galazka, J.M. and Zhang, F., 2018. Developing a Cas9-based tool to engineer native plasmids in Synechocystis sp. PCC 6803. Biotechnology and bioengineering, 115(9), pp.2305-2314.

For PCC 7002 non-essential sites such as A0159 and A2842 may be used, these sites are described in Vogel, A.l.M., Lale, R. and Hohmann-Marriott, M.F., 2017. Streamlining recombination-mediated genetic engineering by validating three neutral integration sites in Synechococcus sp. PCC 7002. Journal of biological engineering, 11(1), p.19.

Non-essential sites suitable for PCC 7942 are described in Kulkarni, R.D. and Golden, S.S., 1997. mRNA stability is regulated by a coding-region element and the unique 5′ untranslated leader sequences of the three Synechococcus psbA transcripts. Molecular microbiology, 24(6), pp.1131-1142; and Andersson, C.R., 2000. Application of bioluminescence to the study of circadian rhythms in cyanobacteria. Methods Enzymol., 305, pp.527-542

In some embodiments the methods described herein can be used to express a gene in trans. There a number of known plasmids that replicate in cyanobacteria and these can be used with the methods described herein. Suitable plasmids may contain a cyanobacterial replicon selected from pDU1SZ, pDU1LZ, PDC1Z, pFDAZ, pANS, pCC5.2, and pAQ1.

In other embodiments the plasmids may be naturally occurring cyanobacterial plasmids engineered to include a desired nucleic acid sequence. Alternatively the plasmid may be replication incompetent and will therefore only persist in a cell if it integrates into the cells genome.

The plasmids may also contain replication origins for commonly used bacteria such as E. coli to facilitate modification of the plasmid sequences, and preparation of the plasmid in an amendable species before transformation into cyanobacteria using the methods disclosed herein.

In some embodiments a promoter is operatively coupled to the sequence of interest (whether in a suicide vector or a replicative vector).

The promoter may be a constitutively active promoter or an inducible promoter. An inducible promoter is one that responds to a specific signal. In some embodiments an inducible promoter will not be activated in the absence of inducer, it will produce a predictable response to a given concentration of inducer or repressor. This response may be binary (i.e., on/off) or graded change with different concentrations of inducer/repressor. Ideally, saturating concentrations of the inducer is not harmful to the cyanobacteria host organism.

The inducible promoter may be a metal inducible promoter, a metabolite inducible promoter, or a macronutrient inducible promoter.

The metal inducible promoter may be selected from the group comprising ArsB (induced by AsO²⁻), ziaA (induced by Cd²⁺ or Zn²⁺), coat (induced by Co²⁺ or Zn²⁺), nrsB (induced by Co²⁺ or Ni²⁺), petE (induced by Cu+²), isiAB (repressed by Fe³⁺), idiA (repressed by Fe²⁺), and Smt (induced by Zn²⁺).

The metabolite inducible promoter may be selected from the group comprising the tetracycline inducible and the IPTG (Isopropyl β-D-1-thiogalactopyranoside) inducible tetR, trp-lac, Trc, A1lacO-1, trc10, trc20, LlacO1, clac143, and Trc. In one embodiment the inducible promoter is clac143.

The macronutrient inducible promoter may be selected from psbA2 (induced by light), psbA1 (induced by light), nirA (induced by NO₃⁻, repressed by NH₄⁺), and Nir (induced by NO₃⁻, repressed by NH₄⁺).

The promoter may be a Type I, Type II or Type III promoter. A type I promoter comprises transcriptional start site at +1 (by definition), a -10 element (consensus sequence 5′-TATAAT-3′), and a -35 element (consensus sequence 5′-TTGACA-3′). A type II promoter is usually used when expression of a gene is to be induced by stress or adaptation responses and thus are normally transcribed by group 2 sigma factors. Type II promoters have a -10 element but typically lack the -35 element. Type III promoters do not have regular -10 and -35 elements. Accordingly, the choice of promoter can be tailored to the desired growth conditions.

In some embodiments a constitutive promoter may be used. Examples of suitable constitutive promoters include cpc560, psbA, plastocyanin promoter, BBaJ23101, and J23.

### Initial Incubation

After the cyanobacteria are contacted with the nucleic acid they are incubated for a period of at least one hour. The incubation period may be at least 1, 2, 3, 4, 5, 6, 7, 8, 9, or 10 hours. In some embodiments the incubation period is 4, 5, or 6 hours, for example 5 hours. In this incubation period the temperature is selected to suit the cyanobacterial strain being transformed and may be in the range of about 15° C. to about 35° C., for example the temperature may be about 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., or about 35° C.

Any light conditions may be used in this initial incubation, for example low light, low-light conditions (i.e. less than 100 µmol photons · m⁻² · s⁻¹), normal light conditions (from 100 - 750 µmol photons · m⁻² · s⁻1) or light saturated conditions (greater than 750 µmol photons · m-2 · s⁻¹). In one embodiment low-light conditions are used.

During the initial incubation the liquid cultures are agitated, for example on a shaker rocker or rotator. Typically, an orbital shaker is used as the shaker. The orbital shaker can utilise a variety of rotation speeds for example from about 10 rpm to about 500 rpm. In some embodiments a rotation speed of about 100 rpm is used.

### Recovery

After this initial incubation period additional growth media is added to the cyanobacteria and they are allowed to recover for a period under culture conditions.

A volume of addition culture medium in excess of the aliquot volume is used, for example a 1, 2, 3, 4, 5, 6, 7, 8, 9, or at least a 10 fold excess of culture medium can be added, limited only by the volume of the container. For example in embodiments where a 20 ul aliquot is use an additional 180 µL media (a 9-fold excess) can be added.

After addition of the excess growth media the cells are incubated for a period of 1-24 hours before a selection pressure is added. The culture time may be 1, 2, 3, 4, 5, 6, 7, 8, 9, 10, 11, 12, 13, 14, 15, 16, 17, 18, 19, 20, 21, 22, 23, or 24 hours. As exemplified herein, in some embodiments the recovery period may be from 4 to 18 hours.

The culture conditions during recovery are selected from the conditions set out above for the initial incubation.

### Selection

After recovery a selection agent is added to the cultures. The selection agent is chosen to correspond to the selection marker of the nucleic acid, for example if the selection marker is a chloramphenicol resistance gene then the selection agent will be chloramphenicol.

The amount of selection agent to be added can be determined by a skilled person using publicly available information. In embodiments where the selection agent is an antibiotic the final concentration typically ranges from about 5 µg/mL to about 500 µg/mL.

The culture conditions during selection are be selected from the conditions set out above for the initial incubation.

At various time points during selection a sample of the culture can be removed and plated on agar plates to assess whether clones resistant to the selection agent (and therefore successfully transformed) have been produced. A convenient initial time point is 48 hours. In other embodiments samples may be taken at about 12 hours, 24 hours, 36 hours, 48 hours, 60 hours, 72 hours, 84 hours, 96 hours, 108 hours, 120 hours, 132 hours, 144 hours or later.

In some embodiments after the sample is taken the remaining culture is topped up with fresh culture media containing the selection agent.

The samples are plated on solid or semi-solid media using media and methods known in the art to allow individual colonies to form.

Once individual colonies have formed they can be tested to assess whether the transformation has been successful and whether the transformants are fully segregated. This can be achieved using PCR with primers directed to the flanking regions, for example if the distance between the flanking regions of the nucleic acid is for example 1.5 kb and the distance between the flanking regions in the genome is 500 bp a simple PCR reaction will be identify whether the individual colonies comprise successfully transformed cyanobacteria and whether the altered chromosomes have fully segregated. A single PCR product will confirm that there is no remaining copies of the ‘wild-type’ chromosome. Alternatively, RT-PCR can be used to assess whether transformants have fully-segregated.

In alternate embodiments the samples can be tested for transformation success and for complete segregation without first allowing individual colonies to form.

### Strains

Most cyanobacteria harbor genes encoding proteins for type IV pili apparatus which are known to be involved in natural competence. Accordingly, it is envisaged that the methods disclosed herein can be used with any genus of cyanobacteria having type IV pili.

Cyanobacterial genera that can be transformed using the methods disclosed herein include those selected from the group comprising Collenia, Girvanella, Gunflintia, Morania, Sphaerocodium, Acaryochloris, Anabaena, Anabaenopsis, Aphanizomenon, Arthrospira, Aulosira, Borzia, Calothrix, Chlorogloeopsis, Chroococcidiopsis, Cyanobacterium, Cyanonephron, Cyanothece, Cylindrospermopsis, Cylindrospermum, Gloeobacter, Gloeocapsa, Gloeotrichia, Homoeothrix, Jakutophyton, Johannesbaptistia, Loefgrenia, Lyngbya, Merismopedia, Microcystis, Nodularia, Nostoc, Oscillatoria, Ozarkcollenia, Palaeolyngbya, Petalonema, Planktothrix, Prochlorococcus, Prochloron, Radaisia, Rivularia, Rothpletzella, Scytonema, Spirulina, Synechococcus, Synechocystis, Trichodesmium, and Wollea.

In some embodiments suitable strains include those to be amendable to genetic modification using traditional methods such as Synechocystis sp. PCC 6803, Synechococcus elongatus PCC 7942, Synechococcus sp. PCC 7002, Synechococcus sp. UTEX 2973, Synechococcus sp. UTEX 3153, Synechococcus sp. UTEX 3154, Anabaena variabilis PCC 7120, and Leptolyngbya sp. BL0902

### Amenability to High Through-Put Approach

The methods disclosed herein utilise relatively small volumes of cells and therefore large numbers of transformations can be carried out in parallel using multi-well plates or the like. This, combined with the relatively short time to isolate transformed and fully-segregated clones, makes the method amenable to automation using commercially available plate, fluid and incubation systems. Accordingly, it is envisaged that the methods disclosed herein can be automated.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

### Example 1: General Method of Transformation and Segregation

Cyanobacteria (Synechocystis sp. PCC 6803, Synechococcus elongatus PCC 7942) were grown to a mid-log phase in BG-11A media which is a modified version of the commonly used BG-11 media 52 mg/L K₂HPO₄ x 3H₂O (as compared to 30 mg/L in BG-11) and 100 mM NaHCO₃ (sodium bicarbonate). In some example AA+ media is used, this media is described in, for example Vogel et al. Journal of Biological Engineering (2017) 11:19. Synechococcus sp. PCC 7002, is grown in AA+.

The cells were then pelleted and washed with 10 mM NaCl before resuspending in fresh BG-11A medium to a density of approximately 10⁹ cells per mL. 20 µL aliquots of the resuspended cells were placed in PCR tubes, 100 ng of DNA was added to each aliquot and gently mixed. The DNA contains the sequences of interest, for example a selectable marker and upstream and downstream flanking regions that are homologous to a portion of the cyanobacterial genome (see examples below for details). The mixtures were then incubated at 30° C. for 5 hours, at 100 rpm under low light conditions, approximately 35 µmol photons • m⁻² · s⁻¹ with broad spectrum white light. Each cell and DNA mixture was then transferred to a 96-well plate and 180 µL BG-11A media was added and the plate was then incubated for a further 18 hours at 30° C., 100 rpm under low light conditions. After the 18 hour incubation, selection agent (chloramphenicol) was added to a final concentration of 25 µg/ml for Synechocystis sp. PCC 6803, Synechococcus sp. PCC 7002 and 5 µg/ml for Synechococcus elongatus PCC 7942. The 96-well plate was then incubated for 144 hours at 30° C., 100 rpm under low light conditions after which a portion of the culture was plated on agar in petri dishes, 6-well or 12-well plates including selection agent at the concentrations noted above.

The steps for transforming and obtaining fully-segregated clones of the transformed cyanobacteria are as follows:
1. Pellet mid-log phase cells, optionally was with 10 mM NaCl.
2. Resuspend cells to approximately 10⁹ cells per mL in growth media.
3. Dispense 20 µL aliquots of the resuspended cells to individual tubes or wells.
4. Add nucleic acid (e.g. 100 ng DNA) to the aliquots.
5. Incubate at 30° C. for 5 hours at 100 rpm under low light conditions.
6. Recovery: add 180 µL media to the aliquots and incubate at 30° C. for 18 hours at 100 rpm under low light conditions.
7. Add selection agent (e.g. 25 µg/ml chloramphenicol for Synechocystis sp. 6803, Synechococcus sp. PCC 7002 and 5 µg/ml chloramphenicol for Synechococcus elongatus PCC 7942).
8. Incubate at 30° C. for 144 hours at 100 rpm under low light conditions.
9. Plate out a portion of the of the culture onto media with a selection agent to obtain individual colonies.

Colonies that contain successfully transformed and fully segregated cyanobacteria can be identified by PCR amplification of the nucleic acid (or a portion thereof) added in step 4 using primers directed to the flanking regions.

This method was followed in the following examples except for the changes noted in each example.

### Example 2: 4 Hour and an 18 Hour Recovery

In this example the method of Example 1 was followed but using both a 4 hour and an alternate 18 hour recovery step to assess whether a shorter recovery time could be used to reduce the time required to obtain transformants. No significant difference in success of the method (production of fully segregated transformed clones) was observed between 4 h and 18 h recovery.

Table 1 sets out the relevant parameters for this Example.

It was also observed that transformed cells grown on non selection media retained the selection marker (Chloramphenicol resistance) and remained transformed and fully segregated (as verified by PCR).

The level of segregation observed in this example was independent of when selection was applied.

TABLE 1

Parameters used in Example 2

| | | | |
|---|---|---|---|
| Conditions | Value | | |
| Strain | 6803 | 7002 | 7942 |
| Cell harvesting OD | 0.568 | 0.34 | 1.134 |
| Recovery time | 4 hours or 18 hours | | |
| Cm concentration (µg/mL) | 25 | 10 | 10 |
| Time to plating (days) | 9 | | |
| Back dilution after x days | 7 | | |
| Time to transformants after plating (days) | 12 | 9 | NA |
| Media | BG-11 | AA+ | BG-11 |
| Plasmids | CORA-312 | CORA-200 | CORA-410 |

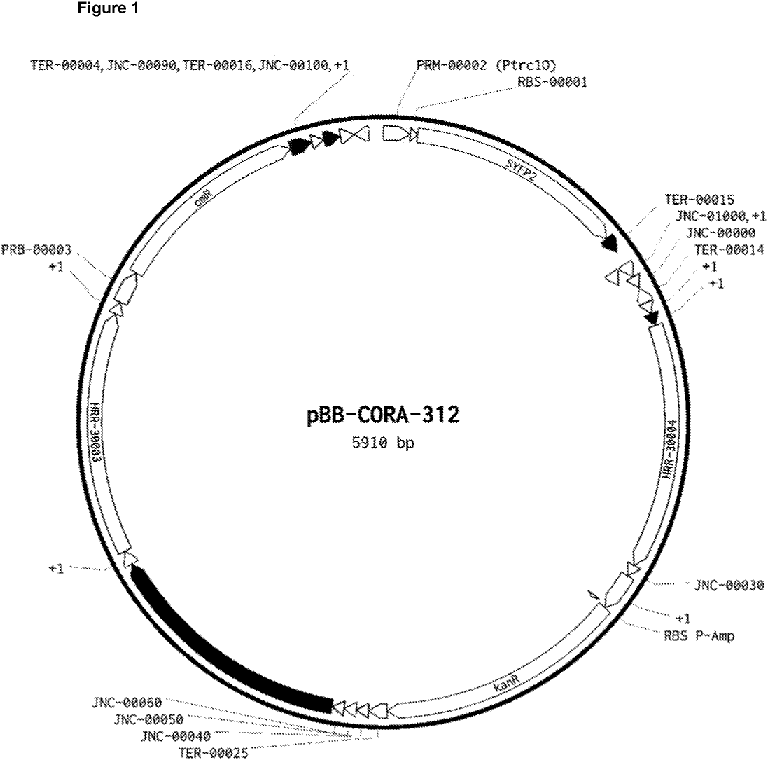

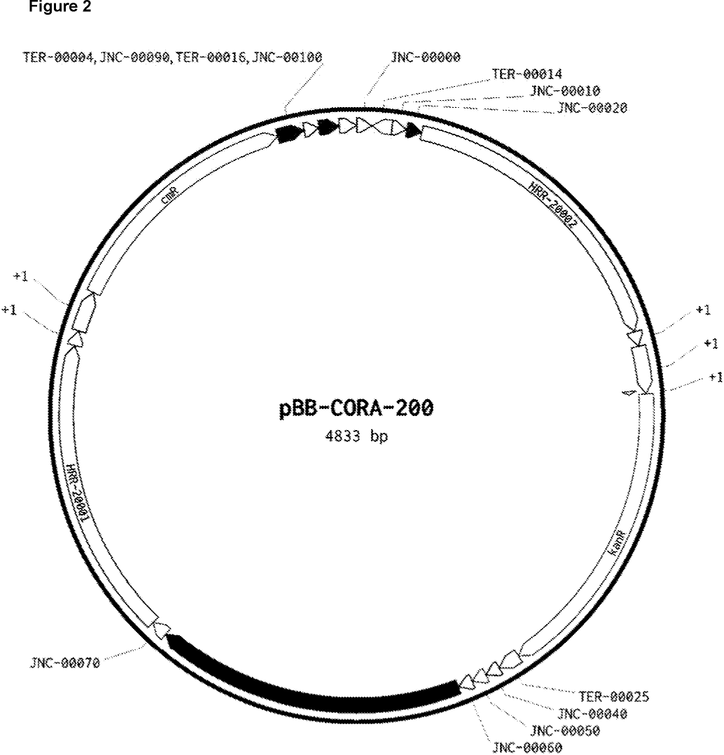

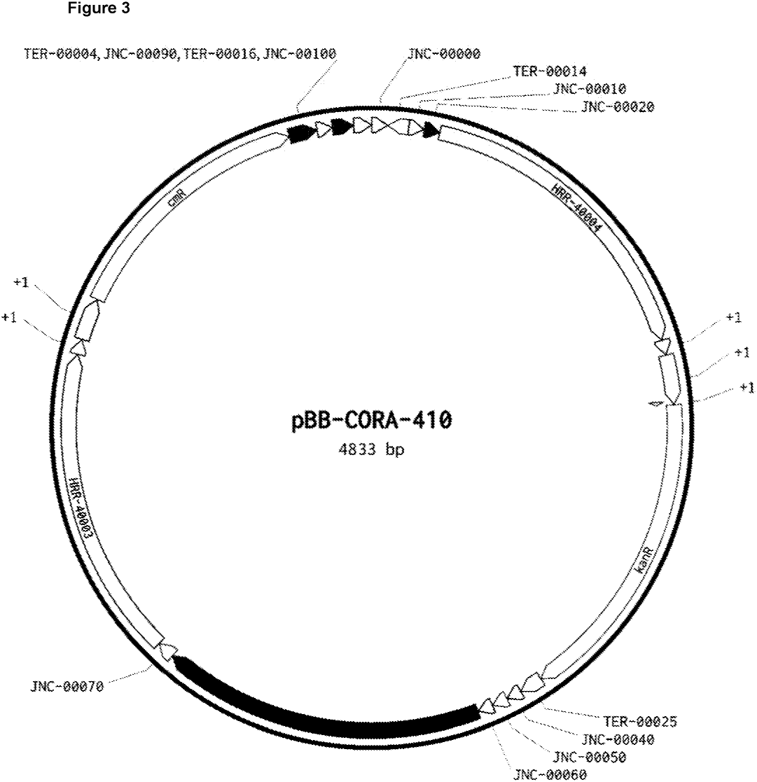

Plasmid maps for CORA-312 (SEQ ID NO: 3), CORA-200 (SEQ ID NO: 1) and CORA-410 (SEQ ID NO: 5) are shown in FIGS. 1-3, respectively.

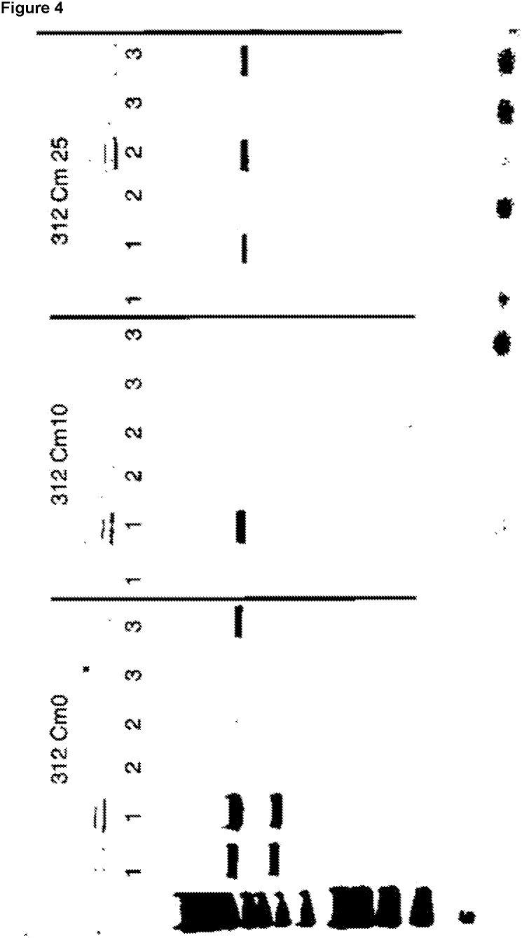

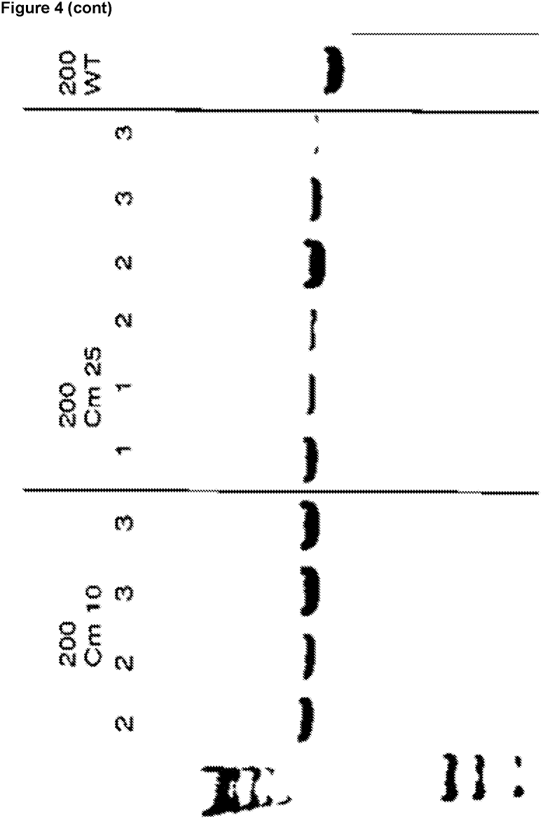

With reference to FIG. 4, for each of the clone numbers, the left lane shows the results of 4 hour recovery while the 18 hour recovery time is shown in the right hand lane. For CORA-312, the example without Cm0 shows partial segregation with both the wild type and transformed chromosomes present. Cm10 and Cm25 show fully segregated colonies.

For CORA-200 there is only the transformant band with no wild type bands in any of the colonies treated with antibiotics and represents fully-segregated clones.

### Example 3: Effect of OD and Time to Plating on Transformation Efficiency of 6803

In this example the OD of the staring culture was varied to assess whether this has an effect on the transformation efficiency. As set out in Table 2, two ODs were chosen to utilise cells at the beginning of exponential phase and one at late exponential phase. Two plating time points were also used.

TABLE 2

Parameters and result for 6803

| | | | | |
|---|---|---|---|---|
| Conditions | Value | | | |
| Strain | 6803 | | | |
| Cell harvesting OD | 0.517 | | 2.948 | |
| Recovery time (hours) | 18 | | | |
| Cm concentration (µg/mL) | 25 | | | |
| Time to plating (hours) | 48 | 144 | 48 | 144 |
| Back dilution after x days | 1:1 after 48 hours | | | |
| Time to transformants after plating (days) | 7 | 3 | 5 | 3 |
| Time from transformation to colonies (days) | 9 | 9 | 7 | 9 |
| No of colonies | Approx. 70 | 3-10 | 25 | 13 |
| Media | BG-11 | | | |
| Plasmids | CORA-312 | | | |

### Example 4: Effect of OD and Time to Plating on Transformation Efficiency of 7002

In this example the OD of the staring culture was varied to assess whether this has an effect on the transformation efficiency. As set out in Table 3, two ODs were chosen to utilise cells at the beginning of exponential phase and one at late exponential phase. Two plating time points were also used.

TABLE 3

Parameters and result for 7002

| | | | | |
|---|---|---|---|---|
| Conditions | Value | | | |
| Strain | 7002 | | | |
| Cell harvesting OD | 0.559 | | 2.612 | |
| Recovery time (hours) | 18 | | | |
| Cm concentration (µg/mL) | 25 | | | |
| Time to plating (hours) | 48 | 144 | 48 | 144 |
| Back dilution after x days | 1:1 after 48 hours | | | |
| Time to transformants after plating (days) | 12 | 7 | 12 | 7 |
| Time from transformation to colonies (days) | 14 | 13 | 14 | 13 |
| No of colonies | 1-2 | 500-1000 | 1-2 | 500 |
| Media | AA+ | | | |
| Plasmids | CORA-200 | | | |

### Example 5: Effect of OD and Time to Plating on Transformation Efficiency of 7002 And 7942

In this example efforts were made to improve transformation efficiency for 7002 and to obtain results for 7942. As set out in Tables 4a, and 4b, three ODs were used for each strain and two timepoints were used for 7942.

TABLE 4a

Parameters and results for 7002

| | |
|---|---|
| Conditions | Value |
| Strain | 7002 |
| Cell harvesting OD | 0.198, 0.387, 0.601 |
| Recovery time (hours) | 18 |
| Cm concentration (µg/mL) | 25 |
| Time to plating (hours) | 144 |
| Back dilution after x days | 1:1 after 48 hours |
| Time to transformants after plating (days) | 2 |
| Time from transformation to colonies (days) | 8 |
| Results | All ODs worked with similar efficiencies, producing lawns containing several thousand colonies |
| Media | AA+ |
| Plasmids | CORA-200 |

TABLE 4b

Parameters and result for 7942

| | | |
|---|---|---|
| Conditions | Value | |
| Strain | 7942 | |
| Cell harvesting OD | 1, 1.19, 1.16 | |
| Recovery time (hours) | 18 | |
| Cm concentration (µg/mL) | 5 | |
| Time to plating (hours) | 18 | 144 |
| Back dilution after x days | 1:1 after 48 hours | |
| Time to transformants after plating (days) | 10 | 5 |
| Time from transformation to colonies (days) | 11 | 11 |
| Results | All ODs worked with similar efficiencies | More colonies on 144 hour plating |
| Media | BG-11 | |
| Plasmids | CORA-402 | |

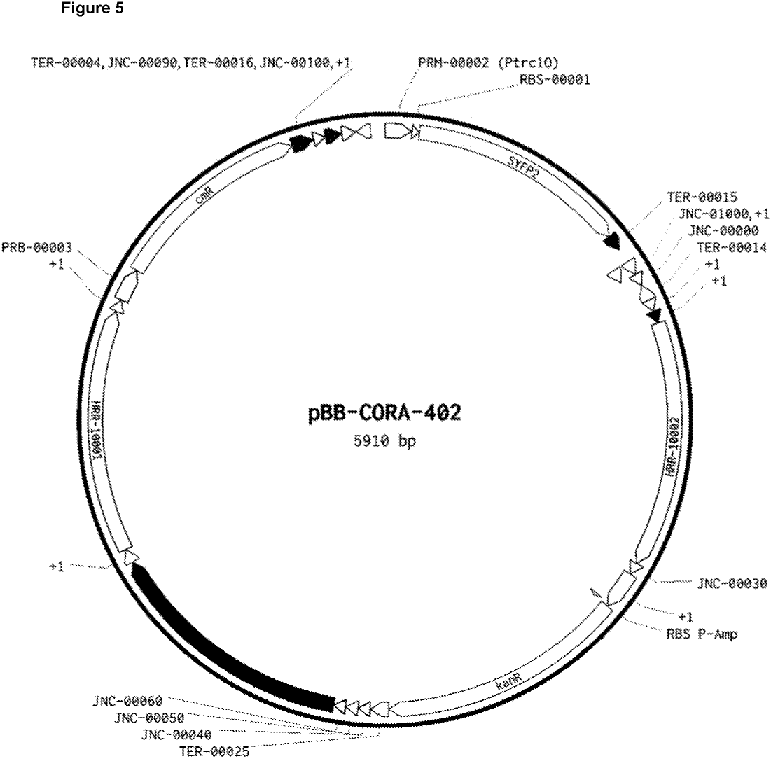

A plasmid map for CORA-402 (SEQ ID NO: 4) is shown in FIG. 5.

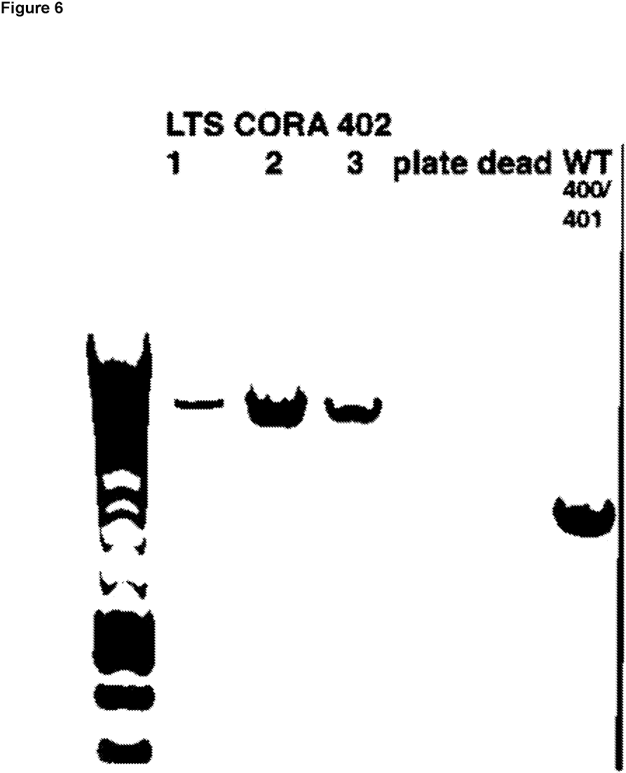

FIG. 6 shows the first fully segregated clone for 7942 after 144 hrs time to plating. Lane 1, cells harvested with an OD of 1. Lane 2, cells harvested with an OD of 1.19. Lane 3, cells harvested with an OD of 1.16.

### Example 6: Effect of DNA Conformation

In this example the CORA-312 and CORA-200 plasmids were used in their circular form to produce linearized DNA via PCR amplification starting and ending with homologous recombination regions and used to transform each of 6803 and 7002.

TABLE 5

Parameters used in Example 6

| | | | | |
|---|---|---|---|---|
| Conditions | Value | | | |
| Strain | 7002 | 6803 | | |
| Cell harvesting OD | 0.266 | 0.446 | | |
| Recovery time | 18 hours | | | |
| Cm concentration (µg/mL) | 25 | 25 | | |
| Time to plating | 144 hours | | | |
| Back dilution after x days | Mixing only after 4 days | | | |
| Time to transformants after plating (days) | 10 | 10 | | |
| Results | Only linear DNA worked | Both linear and plasmid produced colonies. Plasmid worked better | | |
| Media | AA+ | BG-11 | | |
| Plasmids | CORA-200 | | CORA-312 | |
| DNA conformation | Linear | Plasmid | Linear | Plasmid |

7002 and 6803 were successfully transformed with linear DNA (CORA-312 and CORA-200, respectively).

### Example 7: Comparison of DNA Conformation

In this example the circular and linear form of DNA were used to transform 6803. As set out in Table 6, one plasmid was tested

TABLE 6

Parameters used in Example 7

| | | |
|---|---|---|
| Conditions | Value | |
| Strain | 6803 | |
| Cell harvesting OD | 0.447 | |
| Recovery time | 18 hours | |
| Cm concentration (µg/mL) | | 25 |
| Time to plating | 144 hours | |
| Back dilution after x days | Mixing only after 4 days | |
| Time to transformants after plating (days) | 10 | |
| Number of colonies | 200-500 | 500-1000 |
| Results | Both linear and plasmid produced colonies. Plasmid-based showed x2-x3-fold more. | |
| Media | BG-11A | |
| Plasmids | CORA-300 | |
| DNA conformation | Linear | Plasmid |

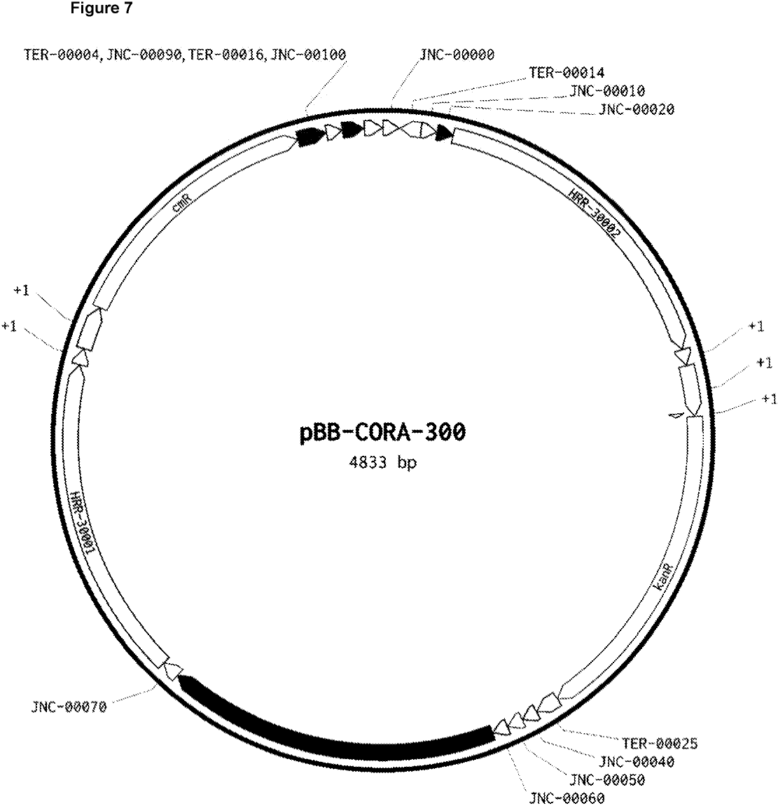

A plasmid map for CORA-300 (SEQ ID NO: 2) is shown in FIG. 7.

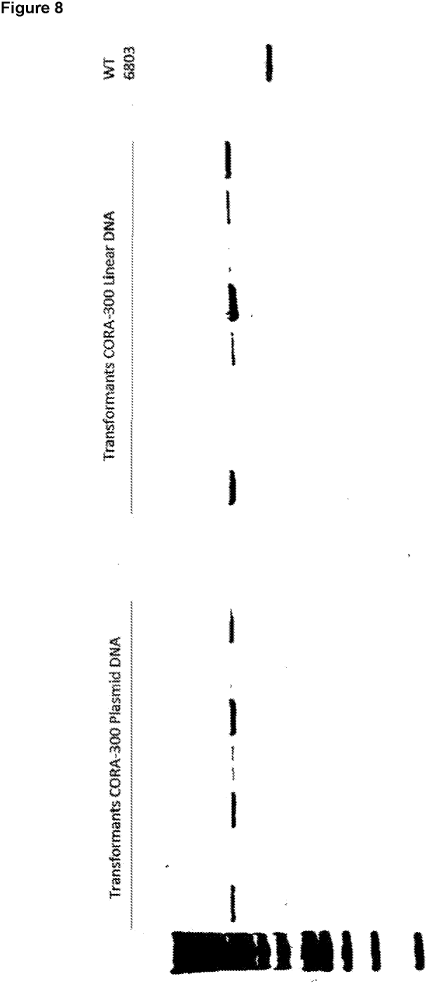

FIG. 8 shows fully segregated colonies of 6803 were successfully transformed with linear and plasmid DNA (CORA-300, respectively). The smaller bands visible for some of the lanes are off-target, misprimed DNA amplifications and represent neither wild type or transformants.

1. A method for producing a fully segregated transformed gram negative micro-organism, the method comprising;
a. incubating the micro-organism and a nucleic acid comprising a selectable marker under conditions suitable for transformation of the micro-organism with the nucleic acid;
b. further incubating the micro-organism in growth media under conditions suitable for recovery of the micro-organism; and
c. selecting the fully segregated transformed micro-organism using a selection agentin liquid media.

2. The method of claim 1 wherein the gram negative micro-organism is a cyanobacteria.

3. The method of claim 2 wherein the cyanobacteria is of the genus Synechococcus or Synechocystis.

4. The method of claim 3 wherein the Synechococcus is Synechococcus sp. PCC 7002,Synechococcus sp.PCC 11901; or Synechococcus elongatus PCC 7942.

5. The method of claim 3 wherein the Synechocystis is Synechocystis sp. 6803.

6. (canceled)

7. The method of claim 2 wherein the cyanobacteria are in an exponential growth phase.

8. The method of claim 7 wherein prior to step a) the cyanobacteria have been cultured in light/dark cycles.

9. The method of claim 8 wherein step a) is performed with the cyanobacteria harvested at or near the end of a light cycle.

10. The method of claim 2 wherein the conditions suitable for transformation comprise incubating the cyanobacteria for a period of 1-10 hours under low light conditions.

11. The method of claim 10 wherein the incubation period is about 5 hours.

12. The method of of claim 2 wherein the conditions suitable for recovery comprise adding growth media and incubating the cyanobacteria for about 1 to about 24 hours under low light conditions.

13. The method of claim 12 wherein the incubation period is about 4 to about 18 hours.

14. The method of claim 2 wherein the selecting comprises adding a selection agent and incubating the cyanobacteria for about 12 to at least about 144 hours under low light conditions.

15. The method of claim 14 wherein the incubation period is about 48 hours to about 144 hours.

16. The method of claim 1 wherein the incubation, further incubation or both are performed in aqueous media.

17. The method of claim 14 further comprising applying a portion of the cyanobacteria to a solid or semi-solid media after the incubation period to obtain individual colonies.

18. (canceled)

19. A method for producing a fully segregated transformed cyanobacteria, the method comprising;
a) incubating the cyanobacteria and a nucleic acid comprising a selectable marker for a period of 1-10 hours under low light conditions;
b) further incubating the cyanobacteria in growth media for about 1 to about 24 hours under low light conditions; and
c) selecting the transformed cyanobacteria using a selection agent, wherein the selecting comprises adding the selection agent and incubating the cyanobacteria for about 12 to at least about 144 hours in liquid media under low light conditions.

20. A method for producing a fully segregated transformed cyanobacteria, the method comprising;
a) incubating the cyanobacteria and a nucleic acid comprising a selectable marker for a period of about 5 hours under low light conditions;
b) further incubating the cyanobacteria in growth media for about 4 to about 18 hours under low light conditions; and
c) selecting the transformed cyanobacteria using a selection agent, wherein the selecting comprises adding the selection agent and incubating the cyanobacteria for about 48 to about 144 hours in liquid media under low light conditions.

21. The method of claim 19 wherein the cyanobacteria are in an exponential growth phase.

22. The method of claim 19 wherein the incubation, further incubation or both are performed in aqueous media.

23. (canceled)
